(19) United States
(12) Patent Application Publication
(10) Pub. No.: US 2009/0185984 A1
(43) Pub. Date: Jul. 23, 2009

(54) COLORANTS SURFACE TREATED WITH URETHANES AND METHODS FOR MAKING AND USING THE SAME

(75) Inventors: Glenn A. Gutkowski, Matawan, NJ (US); William F. Thys, Elmwood Park, NJ (US)

Correspondence Address: MICHAEL BEST & FRIEDRICH LLP, 100 E WISCONSIN AVENUE, Suite 3300, MILWAUKEE, WI, 53202, US

(73) Assignee: SENSIENT COLORS INC., St. Louis, MO (US)

(21) Appl. No.: 12/299,144

(22) PCT Filed: Oct. 12, 2006
(86) PCT No.: PCT/US06/39994

§ 371 Date: Jan. 12, 2009

Related U.S. Application Data
(60) Provisional application No. 60/796,370, filed on May 1, 2006.
(60) Provisional application No. 60/806,189, filed on Jun. 29, 2006.

(51) Int. Cl. A61K 8/87; A61K 8/18; A61Q 1/10; A61Q 1/06; A61Q 17/04; A61Q 11/00

(52) U.S. Cl. 424/49; 424/63; 424/70.7; 424/64; 424/59; 8/405

(57) ABSTRACT

Compositions including colorants surface-treated with silicone polyurethanes, and methods for making surface-treated colorant products, are provided. Cosmetic, . . . personal care, skin care, nail and hair products including the surface treated colorants are disclosed. Also provided are methods for improving the hydrophobicity, or the adherence to the surface of skin, of a colorant. The methods include surface-treating a colorant with an effective amount of a silicone polyurethane to form a surface-treated colorant having an improved hydrophobilicy or an improved adherence to a skin surface compared to the colorant before surface-treatment.

### CROSS-REFERENCE TO RELATED APPLICATIONS

This non-provisional application claims the benefit of U.S. Provisional Application No. 60/796,370, filed on May 1, 2006 and U.S. Provisional Application No. 60/806,189, filed on Jun. 29, 2006, each of which is hereby fully incorporated by reference in its entirety.

### BACKGROUND OF THE INVENTION

Colorants, which include pigments and dyes, are used in a variety of personal care products.

### SUMMARY OF THE INVENTION

In one aspect, the invention provides a composition comprising a colorant surface-treated with an effective amount of a silicone polyurethane. Cosmetic products, personal care products and hair colorants comprising the composition are also provided.

In another aspect, the invention provides a method for making a surface-treated colorant product. The method comprises surface-treating a colorant with an effective amount of a silicone polyurethane to form a surface-treated colorant

In yet another aspect, the invention provides a method of improving the hydrophobicity or the adherence to the surface of skin of a colorant. The method comprises surface-treating a colorant with an effective amount of a silicone polyurethane to form a surface-treated colorant having an improved hydrophobilicy or an improved adherence to a skin surface compared to the colorant before surface-treatment.

### DETAILED DESCRIPTION OF THE INVENTION

Before any embodiments of the invention are explained in detail, it is to be understood that the invention is not limited in its application to the details of construction and the arrangement of components set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The present invention is generally directed to a colorant, such as a pigment, or a color extender, surface treated with a silicone polyurethane, such as a dimethicone/isophorone diisocyanate (dimethicone/IPDI) copolymer, and to a process of treating the surface of an inorganic pigment or organic colorant with a silicone polyurethane. The inventors have discovered that colorants surface treated with a silicone polyurethane may improve at least one of hydrophobicity (i.e. improved waterproof efficacy); adherence to the surface of the skin (including the lips and eyelids); spreadability; and softness and smoothness to the touch. These properties may be improved compared to a colorant not having the silicone polyurethane surface treatment.

As used herein, “surface treating” generally refers to contacting a colorant with an effective amount of a silicone polyurethane.

As used herein, “surface treatment” generally refers to an effective amount of a silicone polyurethane contacted with a colorant.

As used herein, “effective amount of a silicone polyurethane” refers to an amount of a of a silicone polyurethane that, when contacted with a colorant, improves at least one of the following properties of the colorant: hydrophobicity, adherence to the surface of skin, spreadability, softness to touch, smoothness to touch and a combination thereof.

As used herein, the term “colorant” generally refers to a color extender, dye, natural or synthetic inorganic pigment, synthetic organic lake, toner, other agent, or a combination thereof, used to impart a color to a material. In addition, the term “colorant” can include water-soluble and water-insoluble substances, and inorganic or organic colorants, such as pigments or dyes.

As used herein, the term “hydrophobicity,” when referring to a property of a colorant, either surface treated or untreated, generally indicates the ability of the colorant to repel or absorb water. Hydrophilic colorants tend to absorb water, whereas hydrophobic colorants tend to repel water. Hydrophobicity can be determined, for example, by placing drops of water onto pressed colorants, and measuring the time taken for the water to disappear into the colorant. The longer time taken for the water droplet to be absorbed, the more hydrophobic the colorant. Less hydrophobic (hydrophilic) pressed colorants tend to absorb droplets of water placed on their surface quickly, in less than about 20 seconds. More hydrophobic colorants tend to repel droplets of water placed on their surface for more than about 30 seconds.

As used herein, the term “adherence to the surface of the skin,” when referring to a property of a colorant, either surface treated or untreated, generally indicates the ability of a colorant applied to the surface of the skin to resist transfer to another medium. Adherence to the surface of the skin can be measured, for example, by applying the colorant to the surface of the skin and pressing a piece of tissue paper onto the colorant applied to the skin. Most colorant is removed from the skin onto the tissue paper when the colorant's adherence to the surface of skin is low. The amount of colorant transferred from the skin to the tissue paper can, for example, be inspected visually, or measured by weight.

Suitable inorganic colorants for use in the invention include, but are not limited to, at least one of titanium dioxide (e.g., rutile, white or anatase), manganese violet (CI 77742), ultramarine blue, pink or violet (CI 77007), chromium oxide (CI 77288), chromium hydroxide, hydrated chromium oxide (CI 77289), ferric ferrocyanide (e.g., blue), ferric blue (CI 77510), zinc oxide, zirconium dioxide, natural or synthetic iron oxides (e.g., yellow, red, umber, brown, and black), talc, zeolite, kaolin or a combination thereof.

Suitable organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof. Natural organic pigments, including lakes of chlorophyllin copper complex, black carrot extract (anthrocyanin), beet root extracts, riboflavin and curcumin, for example, may also be used.

Suitable organic colorants include, but are not limited to, at least one of various aromatic types including azo, indigoid, triphenylmethane, anthraquinone, bromo, fluorescein and xantheine dyes, dyes which are designated as D&C and FD&C blues, browns, greens, oranges, reds, yellows, natural or synthetic melanin, and insoluble metallic salts of certified color additives, such as aluminum lakes and blends. For example, suitable organic colorants may include D & C Red no. 19 (CI 45170), D & C Red no. 9 (CI 15585), D & C Red no. 21 (CI 45380), D & C Orange no. 4 (CI 15510), D & C Orange no. 5 (CI 45370), D & C Red no. 27 (CI 45410), D & C Red no. 13 (CI 15630), D & C Red no. 7 (CI 15850:1), D & C Red no. 6 (CI 15850:2), D & C Yellow no. 5 (CI 19140), FD & C Yellow no. 5, D & C Red no. 36 (CI 12085), D & C Orange no. 10 (CI 45425), D & C Yellow no. 6 (CI 15985), FD & C Yellow no. 6, D & C Red no. 30 (CI 73360), D & C Red no. 3 (CI 45430), D&C Black no. 2 carbon black (CI 77266), cochineal carmine lake (CI 75470), D & C Yellow no. 10, D & C Red no. 28, D & C Red no. 33, D & C Red no. 40, D & C Green no. 6, D & C Blue no. 1, FD & C Blue no. 1 or a combination thereof.

Examples of color extenders suitable for use in the present invention include white or non-pigmentitious powders such as bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof.

As used herein, the term “payoff” generally refers to the amount of the cosmetic substance transferred to the area of use, usually skin, from the cosmetic as packaged, by whatever means are appropriate (i.e. fingertip, sponge, brush, etc.) and which adheres generally to the area of application.

As used herein, the term “masstone” generally refers to the appearance of a thick layer of a colorant either dry or when incorporated into a vehicle such as water or castor oil. For example, the masstone of a cosmetic product can generally refer to the appearance of the product prior to application of the product.

As used herein, the term “lake” generally refers to a colorant that is prepared by precipitating a soluble dye onto an insoluble reactive or adsorptive substratum or diluent.

The silicone polyurethane can be either water soluble or water insoluble. A suitable water-soluble silicone polyurethane is polydimethyl siloxane-PEG ether/isophorone diisocyanate copolymer. A suitable water insoluble silicone polyurethane is polydimethyl siloxane-PPG ether/isophorone diisocyanate copolymer. Another suitable silicone polyurethane useful herein includes, but is not limited to, dimethiconol/isophorone diisocyanate copolymer. Dimethiconol is a dimethicone copolyol, a polymer of dimethylsiloxane with polyoxyethylene and/or polyoxypropylene side chains.

For example, suitable dimethiconol copolymers include dimethiconol/isophorone diisocyanate copolymer, dimethiconol/isophorone diisocyanate copolymer 50%, dimethiconol-PEG-2 Soyamine/isophorone diisocyanate copolymer, dimethicone copolyol/isophorone diisocyanate copolymer. Other suitable silicone polyurethanes useful herein include dimethicone/isophorone diisocyanate copolymer, and dimethicone/PEG-2 Soyamine/isophorone diisocyanate copolymer.

Suitable polymers are disclosed in U.S. Pat. Nos. 6,800,716, 6,613,866, 6,583,106, 6,392,087, 6,258,348, 6,107,352, 5,972,324, 5,707,612, 5,116,604, and 4,537,762, each of which are hereby incorporated by reference in their entireties. The molecular weights of the silicone polyurethane can vary widely, e.g., from about 2,000 to about 10,000,000. In one embodiment, the molecular weight may be from about 5,000 to about 1,000,000. A suitable silicone polyurethane has the structure:

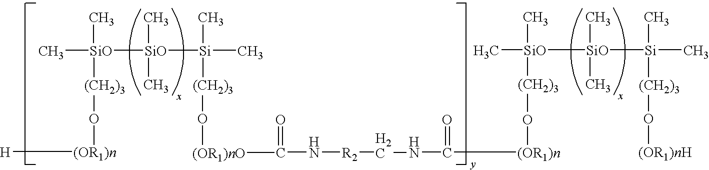

wherein:

R₁ is a C2 to C8 alkylene group, particularly a C2 to C6 alkylene group, more particularly a C2 to C4 unbranched alkylene group, even more particularly a C2 to C3 alkylene or a mixture of C2 and C3 alkylene groups (particularly as polyethylene-co-polypropylene oxide blocks) and n ranges from about 2 to about 200, more particularly about 2 to about 150. Suitably, R₁ is C2 (ethylene oxide);

R₂ is a C2 through C36 (particularly, C6 through C22) linear, cyclic or branch-chained saturated or unsaturated hydrocarbon group which is substituted or unsubstituted, monomeric or dimeric, an aromatic group, including a phenyl or benzyl group or substituted phenyl or benzyl group, an alkylphenyl, alkylbenzyl or substituted alkylphenyl or alkylbenzyl group; or saturated, unsaturated, aromatic or halogen substituted, linear, cyclic, aromatic or branch chained hydrocarbons;

y is an integer from about 1 to about 1000, particularly from about 2 to about 150, more particularly from about 2 to about 20, even more particularly from about 2 to about 10, and yet more particularly from about 2 to about 4;

x is an integer from about 1 to about 9000, particularly about 1 to about 2000, more particularly about 1 to about 1000, and even more particularly about 1 to 250;

A suitable silicone polyurethane has the following structure:

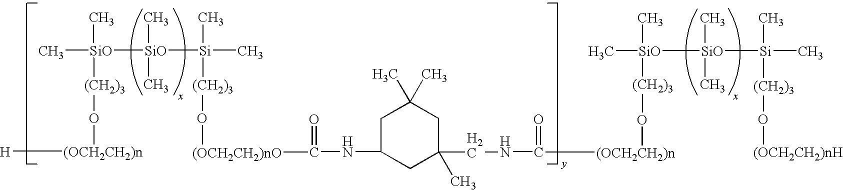

wherein:

y is an integer from about 1 to about 1000, particularly from about 2 to about 150, more particularly from about 2 to about 20, even more particularly from about 2 to about 10, and yet more particularly from about 2 to about 4;

x is an integer from about 1 to about 9000, particularly about 1 to about 2000, more particularly about 1 to about 1000, and even more particularly about 1 to 250;

n ranges from about 2 to about 200, more particularly about 2 to 150.

Other dimethicone copolyol compounds can be employed in the IPDI copolymer used in the present invention, more specifically, those compounds containing the following repeating structure in the siloxane moiety and also containing a reactive hydroxyl group:

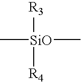

wherein R₃ and R₄ are each, independently, alkyl or aryl.

The R₃ and R₄ groups on the silicone atom may represent the same group or different groups. Suitably, the R₃ and R₄ groups represent the same group. Suitable R₃ or R₄ groups include methyl, ethyl, propyl, phenyl, methylphenyl and phenylmethyl, alpha olefin, allyl alcohol alkoxylated, fluoro compounds and allyl alcohols. Examples of suitable siloxanes are polydimethyl siloxane, polydiethylsiloxane, and polymethylphenylsiloxane. Polydimethylsiloxane is particularly suitable.

The ratio of silicone to diisocyanate in the polyurethane silicone polymer can vary. Suitable ratios of silicone:diisocyanate include from about 1:20 to about 20:1.

The silicone polyurethanes suitable for use in the invention can be prepared by conventional techniques known in the polymer art using polymerization techniques starting with desired silicones and diisocyanates. Other raw materials may also be added to the reaction mixture to form a complex polymer that provides the benefits of the silicone as well as the added raw material. Examples of these added raw materials include tertiary amines (such as those described in U.S. Pat. No. 6,613,866), monohydric alcohols (as described in U.S. Pat. No. 5,972,324), alkanolamine fatty acid esters (as described in U.S. Pat. No. 6,800,716), and castor oil (as described in U.S. Pat. No. 5,707,612). Each of these patents is hereby fully incorporated by reference in their entireties.

The ratio of dimethicone to isophorone diisocyanate in the polyurethane may vary. Suitable ratios include from about 2:1 to about 16:1.

The polyurethanes suitable for use in the invention may be linked to one or more reactive products to further improve the characteristics of the surface treated colorants. For example, the silicone may be an alkyl silane such as triethoxycaprylyl silane, fluorinated silanes, or a dimethicone copolyol.

The colorants may be surface treated by processes known in the art such as spraying or atomizing the surface treatment chemical onto the finely divided surface of the colorant. The mass of colorant is suitably agitated by air or other mechanical means during application of the surface treatment chemical.

The surface treatment can be applied at ratios ranging from 15 parts surface treatment to 85 parts colorant, to 1 part surface treatment to 99 parts colorant. Suitably, the surface treatment can be applied at ratios ranging from 10 parts surface treatment to 90 parts colorant, to 1 part surface treatment to 99 parts colorant. Water may be optionally applied prior to the application of the surface treatment. Water may be used in the ratio of from about 1 part water to about 1 part surface treatment by weight, up to about 1 part water to about 10 parts surface treatment. The colorant so treated may then be dried, for example in an oven, to drive off or remove most or essentially all the free water.

Cationic materials, such as soy amines, may be incorporated into the reaction mixture of the polyurethane:silicone polymer, to improve the adhesion of the treated colorants to the skin.

The surface treatment imparts a smooth velvety feel to the colorant. In water-in-silicone or water-in-oil emulsions, the treated colorant reduces the viscosity of the final formulation, improving spreadability of the emulsion during application to the skin. In pressed powder, the surface-treated colorant improves adhesion of the powdered cosmetic to the skin. The surface-treated colorant also provides advantages in that it improves the cohesiveness of the pressed powder, allowing less binder to be used in the formulation and/or lower compression forces to be used during formation of the pressed powder.

The surface treated colorants can be used in a variety of cosmetic and skin care applications. For example, cosmetic products, personal care products, hair care products, paints, inks, plastics, leather and other surface treatments, other applications in the automobile industry, other applications in the printing industry, and combinations thereof.

Cosmetic products can include, but are not limited to, mascaras, pressed powder make-ups (e.g., eye shadows, cheek rouge and facial powders), liquid make-ups (e.g., eye shadows, foundations, cheek rouge, blushes, lip liners, eye liners and nail enamel), lipsticks and other cosmetics made using silicones, or combinations thereof. Personal care products can include, but are not limited to, lotions, creams, gels, toothpastes or combinations thereof.

The surface treated colorants can be combined with a variety of other components to form one or more of the products listed above. Additional components may include, but are not limited to, at least one of other colorants, water soluble sunscreens (such as Eusolex 232); oil soluble sunscreens (such as octyl methoxycinnamate); and organic sunscreens (such as camphor derivatives, cinnamates, salicylates, benzophenones, triazines, PABA derivatives, diphenylacrylate derivatives, and dibenzoylmethane derivatives.); antioxidants (such as BHT); chelating agents (such as disodium EDTA); emulsion stabilizers (such as carbomer); preservatives (such as methyl paraben); fragrances (such as pinene); flavoring agents (such as sorbitol); humectants (such as glycerine); waterproofing agents (such as PVP/Eicosene copolymer); water soluble film-formers (such as hydroxypropyl methylcellulose); oil-soluble film formers (such as hydrogenated C-9 Resin); moisturizing agents, such as cholesterol; cationic polymers (such as Polyquatemium 10); anionic polymers (such as xanthan gum); pigment wetting agents, such as Arlacel™ P100 (polyhydroxystearic acid), or Emerest™ 2452 (Polyglyceryl-3 Diisostearate); vitamins (such as tocopherol); or combinations thereof.

Examples of other components that can be combined with the surface treated colorants to form one or more of the products listed above include, but are not limited to, fats and oils, waxes, surfactants, oxidation inhibitors, UV absorbers, vitamins, hormones, squalenes, liquid paraffins, fatty acids, bees wax, myristyl myristate and other esters, acetone, toluene, butyl acetate, acetic ester and other solvents; antioxidants, antiseptic agents, polyhydric alcohols, perfumes, or combinations thereof.

Examples of other colorants that can be used in combination with the surface treated colorants to form a variety of products, include, but are not limited to, titanium dioxide; calcium carbonate; clay; talc; barium sulfate; white carbon; chromium oxide; zinc oxide; zinc sulfide; zinc powder; metal powder pigments; iron black; yellow iron oxide; red iron oxide; chrome yellow; carbon black; molybdate orange; Prussian Blue; ultramarine blue; cadmium type pigments; fluorescent pigments; soluble azo dyes; insoluble azo dyes; condensed azo dyes; phthalocyanine pigments; condensed polycyclic pigments; composite oxide pigments; graphite; mica (such as, muscovite, phlogopite, synthetic mica, and fluorine tetra silicon mica); metal oxide coated mica (such as titanium oxide coated mica, titanium dioxide coated mica, (hydrated) iron oxide coated mica, mica coated with iron oxides and titanium oxides, mica coated with lower ordered titanium oxides); metal oxide coated graphite (such as titanium dioxide coated graphite); thin platelet-like alumina; metal oxide coated thin platelet-like alumina (such as titanium dioxide coated thin platelet-like alumina, iron oxide coated thin platelet-like alumina, Fe₂O₃ coated thin platelet-like alumina, Fe₃O₄ coated thin platelet-like alumina, interference color metal oxide coated thin platelet-like alumina); metal flake pigments (such as aluminum flakes, colored aluminum flakes, stainless steel flakes, titanium flakes); anti-corrosive metal flakes (such as base flakes of aluminum flake substrates); metal oxide coated metal flakes (such as titanium oxide or iron oxide coated aluminum flakes); MIO; metal oxide coated MIO; metal oxide coated silica flakes and metal oxide coated glass flakes called optical effect pigments (effect pigments); photochromic pigments; thermochromic pigments; holographic pigments; sericite; magnesium carbonate; silica; zeolite; hydroxyapatite; chromium oxide; cobalt titanate; glass beads; nylon beads; silicone beads; red nos. 2, 3, 102, 104, 105, 106, 201, 202, 203, 204, 205, 206, 207, 208, 213, 214, 215, 218, 219, 220, 221, 223, 225, 226, 227, 228, 230-1, 230-2, 231, 232, 405; yellow nos. 4, 5, 201, 202-1, 202-2, 203, 204, 205, 401, 402, 403, 404, 405, 406, 407; green nos. 3, 201, 202, 204, 205, 401, 402; blue nos. 1, 2, 201, 202, 203, 204, 205, 403, 404; orange nos. 201, 203, 204, 205, 206, 207, 401, 402, 403; brown no. 201; violet nos. 201, 401; black no. 401; salol yellow; carmine; beta-carotin; hibiscus color; capsaicin; carminic acid; laccaic acid; gurcumin; riboflavin; shikonin; or a combination thereof.

Other products may comprise a surface treated colorant and one or more active components. Examples of active components can include, but are not limited to, those that improve or eradicate age spots, keratoses and wrinkles, anti-cavity ingredients for oral care and toothpaste products, such as fluoride, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, skin lightening agents, depigmenting agents, wound-healing agents, vitamins, corticosteroids, tanning agents, sunscreens, hormones, or combinations thereof. Examples of active compounds can further include, but are not limited to, retinoids, such as retinol, and esters, acids, and aldehydes thereof; ascorbic acid, and esters and metal salts thereof; tocopherol and esters and amide derivatives thereof; shark cartilage; milk proteins; alpha- or beta-hydroxy acids; DHEA and derivatives thereof; topical cardiovascular agents; or combinations thereof. Examples of active compounds can also include, but are not limited to, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, hydrocortisone, hydrocortisone 21-acetate, hydrocortisone 17-valerate, hydrocortisone 17-butyrate, betamethasone valerate, betamethasone diproprionate, triaminolone acetonide, fluocinonide, clobetasol, proprionate, benzoyl peroxide, crotamiton, propranolol, promethazine, or a combination thereof.

Various features and aspects of the invention are set forth in the following examples.

### Example 1

### Colorant Composition (Polyderm/Titanium Dioxide)

The following ingredients were used:

| | |
|---|---|
| Titanium dioxide | 47.5 g |
| Deionized water | 2 g |
| Polyderm (BIS-PEG-1 Dimethicone/IPDI Copolymer; | 2.5 g |
| L-2) (available from Alzo Int'l, Sayreville, NJ) | |

The titanium dioxide and water were mixed for 30 seconds in a kitchen-type blender and the mixture was brushed down from the sides of the container and blades before manually mixing. The Polyderm (BIS-PEG-1 Dimethicone/IPDI Copolymer) was added in thirds, with a 30 second mix and brush down after each addition. The batch was heated overnight in an 80° C. oven. The percentage loss on drying was 0.38%. The bulk density was 6.5 g/cubic inch. The hydrophobicity was tested on still water and shaken water and found to be very good.

### Example 2

### Colorant Composition (Polyderm/Talc)

The following ingredients were used:

| | |
|---|---|
| Talc | 47.5 g |
| Deionized water | 1 g |
| Polyderm (BIS-PEG-1 Dimethicone/IPDI Copolymer; | 2.5 g |
| L-2) (available from Alzo Int'l, Sayreville, NJ) | |

The talc and water were mixed for 30 seconds in a kitchen-type blender and the mixture was brushed down from the sides of the container and blades before manually mixing. The Polyderm (BIS-PEG-1 Dimethicone/IPDI Copolymer) was added in thirds, with a 30 second mix and brush down after each addition. The batch was heated overnight in an 80° C. oven. The percentage loss on drying was 0.72%. The bulk density was 6.4 g/cubic inch. The hydrophobicity was tested on still water and shaken water and found to be very good.

### Example 3

### Colorant Composition (Polyderm/Red Iron Oxide)

The following ingredients were used:

| | |
|---|---|
| Unipure Red LC 383 Iron Oxide (available from | 47.5 g |
| Sensient Cosmetic Technologies, (LCW), South | |
| Plainfield, NJ) | |
| Deionized water | 1 g |
| Polyderm (BIS-PEG-1 Dimethicone/IPDI Copolymer; | 2.5 g |
| L-2) (available from Alzo Int'l, Sayreville, NJ) | |

The red iron oxide and water were mixed for 30 seconds in a kitchen-type blender and powder brushed down from the sides of the container and blades before manual mixing. The Polyderm (BIS-PEG-1 Dimethicone/IPDI Copolymer) was added in thirds, with a 30 second mix and brush down after each addition. The batch was heated overnight in an 80° C. oven. The percentage loss on drying was 0.36%. The bulk density was 10.7 g/cubic inch. The pigment showed good hydrophobicity on still water for one hour. After vigorously shaking the pigment in water and allowing the mixture to stand for one hour, a clear solution resulted with a trace of suspended fine particles, a top floating layer and a bottom precipitate. The skin feel of the treated pigment was very soft, and showed high adhesiveness to skin under running water.

### Example 4

### Performance of Treated Materials in an Anhydrous Nail Lacquer

| Phase A | |
|---|---|
| Nail Polish Base: | % w/w |
| Nitrocellulose | 13.2 |
| Dibutyl phthalate | 4.0 |
| Toluene sulfonamide formaldehyde resin | 5.3 |
| Butyl acetate | 26.4 |
| Ethyl acetate | 7.0 |
| Toluene | 22.9 |
| Isopropanol | 6.5 |
| Bentone 27 | 0.9 |
| COVAICE ™ (optical modifier; available | 5.8 |
| from Sensient Cosmetic Technologies, (LCW), | |
| South Plainfield, NJ) | |
| Pigment made in Example 3 | 8.0 |
| | 100.0% |

The material made in Example 3 is dispersed in a small amount of nail polish base until homogenous using a propeller mixer. The pigment dispersion of Example 3 is added to the nail polish base and is blended for 5 minutes. Mixing is continued until a homogeneous blend is achieved. COVAICE™ (optical modifier; available from Sensient Cosmetic Technologies, (LCW), South Plainfield, N.J.) is slowly added to the bulk blend to make the colors even. The blend is placed into an appropriate container, suitably a clear container.

### Example 5

### Performance of Treated Material in an Aqueous Nail Lacquer

| | % W/W |
|---|---|
| PHASE A | |
| COVACRYL ™ MS 11 (available from | 82.70 |
| Sensient Cosmetic Technologies, (LCW), | |
| South Plainfield, NJ) | |
| COVAPLAST ™ (available from Sensient | 4.70 |
| Cosmetic Technologies, (LCW), South | |
| Plainfield, NJ) | |
| PHASE B | |
| Pure Water | 7.85 |
| COVACRYL ™ J22 (available from Sensient | 0.65 |
| Cosmetic Technologies, (LCW), South | |
| Plainfield, NJ) | |
| PHASE C | |
| Glycerin | 2.50 |
| Pigment made in Example 3 | 1.60 |
| | 100.00% |

The pigment of Phase C is dissolved in glycerin and dispersed using a three-roller mill. Phase A components are mixed together and set aside. Covacryl J22 is dissolved in water and added to Phase C. While mixing, Phase B & C are slowly added into A, and mixed until the color is homogeneous.

### Example 6

### Performance of Treated Materials in Liquid Foundation

| | % w/w |
|---|---|
| Phase A | |
| SILAMER (Phenyl trimethicone & cetyl PEG/PPG-10/1 dimethicone | 15.00 |
| & polyglyceryl-2-isostearate & hexyl laurate (available from Sensient | |
| Cosmetic Technologies (LCW), South Plainfield, NJ) | |
| Cetyl Dimethicone (Abil Wax 9801; available from Degussa Corp., | 1.90 |
| Parsippany, NJ) | |
| Synthetic Beeswax | 0.65 |
| Hydrogenated Castor oil (castor wax; available from CasChem) | 0.50 |
| Hexyl Laurate (Cetiol A, available from Cognis Care Chemicals) | 2.70 |
| Phase B | |
| TITANIUM DIOXIDE 5% Polyderm (BIS-PEG-1 Dimethicone/IPDI | 7.50 |
| Copolymer) (made in Example 1) | |
| RED IRON OXIDE LC 383 5% Polyderm (BIS-PEG-1 | 1.55 |
| Dimethicone/IPDI Copolymer) (made in Example 3) | |
| LUMIRAL (light diffusing pigment; available from Sensient Cosmetic | 1.20 |
| Technologies (LCW), South Plainfield, NJ) | |
| COVABEAD GLASS (spherical powder; available from Sensient | 4.60 |
| Cosmetic Technologies (LCW), South Plainfield, NJ) | |
| Phase C | |
| Dow Corning 245 (cyclomethicone, available from Dow Corning) | 10.00 |
| Phase D | |
| D.I. Water | 47.40 |
| Polyglycerol-3 Laurate (Hydramol TGL, available from Noveon Inc.) | 0.40 |
| Propylene Glycol (Propylene Glycol USP/EP, available from Dow Chemical) | 6.00 |
| Preservative: Germaben II (propylene glycol, diazolidinyl urea, | 0.40 |
| methylparaben and propylparabens, available from Sutton | |
| Laboratories) | |
| | 100.00% |

The components of Phase A were mixed and heated to 80° C. The components of Phase B were blended until homogenous. Phase B was added to Phase A, and mixed for 15 minutes using a propeller mixer until homogenous. Phase C was added and mixed for 5 minutes. Phase D was added at 70° C. The batch was homogenized for one hour at up to 45° C. temperature. Dispersion of the batch was checked. The batch was cooled and placed into an appropriate container.

The viscosity of this formulation was found to be 4200 cP (Brookfield spindle # LV3 (S63) at 12 rpm). A comparable formulation made with red iron oxide and titanium dioxide that had been treated with triethoxycaprylyl silane showed a viscosity of 7300 cP (Brookfield spindle # LV4 (S64) at 30 rpm).

### Example 7

### Performance of Treated Colorants in Pressed Powders

| | % w/w |
|---|---|
| Phase A | |
| Talc 141 | 78.5 |
| Magnesium stearate (available from Spectrum Chemical) | 2.0 |
| Preservative: Propylparaben (available from Protameen | 0.50 |
| Chemical Inc.) | |
| Phase B | |
| Red Iron Oxide LC 383 w/ 5% Polyderm (BIS-PEG-1 | 10.0 |
| Dimethicone/IPDI Copolymer) + H₂O (made in Example 3) | |
| Phase C | |
| COVAPRESS TW 966 (binding agent; available from Sensient | 9.0 |
| Cosmetic Technologies (LCW), South Plainfield, NJ) | |

Phase A and B were processed in a blender twice for 30 seconds each time until the color was homogeneous. The sides of the container were tapped and scraped to remove the blend. Phase C was added, mixed with a spatula and processed again for another 2 minutes. Two (2) grams of the blend were weighed in a metal pan and pressed in a Carver press with a pressure of 340 psi. The resulting compact compressed at 340 psi was still intact after being dropped three times from a height of 12 inches.

### Example 8

### Performance of Untreated Colorants in Pressed Powders

| | % w/w |
|---|---|
| Phase A | |
| Talc 141 | 71.5 |
| Magnesium stearate (available from Spectrum Chemical) | 5.0 |
| Preservative: Propylparaben (available from Protameen | 0.50 |
| Chemical Inc.) | |
| Phase B | |
| Red Iron Oxide LC 383 w/ 5% Polyderm (BIS-PEG-1 | 10.0 |
| Dimethicone/IPDI Copolymer; L-2) + H₂O (made in Example 3) | |
| Phase C | |
| COVAPRESS TW 966 (binding agent; available from Sensient | 13.0 |
| Cosmetic Technologies (LCW), South Plainfield, NJ) | |

Phase A and B were processed in a blender twice for 30 seconds each time until the color was homogeneous. The sides of the container were tapped and scraped to remove the blend. Phase C was added, mixed with a spatula and processed again for another 2 minutes. Two (2) grams of the blend were weighed in a metal pan and pressed in a Carver press with a pressure of 340 psi. The resulting compact compressed at 340 psi cracked on the third drop after being dropped three times from a height of 12 inches. In addition, the compact containing untreated pigment required 44% more binder than the compact containing the treated pigment in order to form a cohesive mass.

### Example 9

### Treated Titanium Dioxide

The following ingredients were used to make a 50 g batch:

| | |
|---|---|
| Ultrafine titanium dioxide | 47.5 g |
| Deionized water | 1 g |
| Polyderm (BIS-PEG-1 Dimethicone/IPDI Copolymer | 2.5 g |
| L-2) (available from Alzo Int'l, Sayreville, NJ) | |

The ultrafine titanium dioxide and water were mixed for 30 seconds in a kitchen-type blender and the powder brushed down from the sides of the container and blades before manual mixing. The Polyderm (BIS-PEG-1 Dimethicone/IPDI Copolymer) was added in half portions, with a 30 second mix and brushdown after each addition. An additional 30 second mixing was performed after the last addition of Polyderm (BIS-PEG-1 Dimethicone/IPDI Copolymer), with an additional final brushdown. The batch was easy to stir and did not stick to the glass. The batch was fluffy and light. The batch was heated overnight in an 80° C. oven. Bulk density of the batch was 2.5 g/cu. in and loss on drying was 0.76%. The spreadability was good and the adhesiveness to skin under running water was moderately high. The skin feel was gritty, with a slight drag in the smoothness. The hydrophobicity was tested on still water and there was immediate gradual precipitation, with top layer containing a lot of powder over a cloudy white solution.

### Example 10

### Treated Titanium-Dioxide

The following ingredients were used to make a 50 g batch:

| | |
|---|---|
| Ultrafine titanium dioxide | 45 g |
| Deionized water | 2 g |
| Polyderm (BIS-PEG-1 Dimethicone/IPDI Copolymer; | 5 g |
| L-2) (available from Alzo Int'l, Sayreville, NJ) | |

The ultrafine titanium dioxide and water were mixed for 30 seconds in a kitchen-type blender and the powder was brushed down from the sides of the container and blades before manually mixing. The Polyderm (BIS-PEG-1 Dimethicone/IPDI Copolymer) was added in half portions, with a 30 second mix and brushdown after each addition. An additional 30 second mixing was performed after the last addition of Polyderm (BIS-PEG-1 Dimethicone/IPDI Copolymer), with an additional final brushdown. The batch was easy to stir and did not stick to the glass. The batch was fluffy and light. The batch was heated overnight in an 80° C. oven. Bulk density of the batch was 4.2 g/cu. in, and moisture content was 0.64%. The batch exhibited hydrophobicity for more than one hour on still water. The spreadability was good and adhesiveness of the product to skin under running water was very high and stronger than Example 9. The skin feel was smoother than the material made in Example 9.

### Example 11

### Treated Titanium Dioxide

The following ingredients were used to make a 50 g batch:

| | |
|---|---|
| Ultrafine titanium dioxide | 42.5 g |
| Deionized water | 3 g |
| Polyderm (BIS-PEG-1 Dimethicone/IPDI Copolymer) (available | 7.5 g |
| from Alzo Int'I, Sayreville, NJ) | |

The ultrafine titanium dioxide and water were mixed for 30 seconds in a kitchen-type blender and powder brushed down from the sides of the container and blades before manual mixing. The Polyderm (BIS-PEG-1 Dimethicone/IPDI Copolymer) was added in half portions, with a 30 second mix and brushdown after each addition. An additional 30 second mixing was performed after the last addition of Polyderm (BIS-PEG-1 Dimethicone/IPDI Copolymer), with an additional final brushdown. The batch was heated overnight in an 80° C. oven. Bulk density of the batch was 6.1 g/cu. in, and moisture content was 0.48%. The batch exhibited hydrophobicity for more than one hour on still and shaken water.

### Example 12

The material made according to Example 11 is made into a sunscreen according to the following formula:

| | % w/w |
|---|---|
| Phase A | |
| Pure Water | 68.00 |
| Glycerin | 10.00 |
| Material made in Example 11 | 8.00 |
| Preservative (Germaben II, Propylene glycol, diazolidinyl urea, | q.s. |
| methyl paraben and propylparabens, available from Sutton | |
| Laboratories) | |
| Phase B | |
| BASE O/W 097 (emulsifying base; available from Sensient | 8.00 |
| Cosmetic Technologies, (LCW), South Plainfield, NJ) | |
| Phase C | |
| COVASTEROL (a plant-based lanolin substitute; available from | 2.00 |
| Sensient Cosmetic Technologies, (LCW), South Plainfield, NJ) | |
| SQUATOL S (a hydrogenated polyisobutene; available from | 4.00 |
| Sensient Cosmetic Technologies, (LCW), South Plainfield, NJ) | |
| | 100.00 |

Phase A components are heated to 40° C. and mixed using a propeller blade mixer until homogenous. Phase B and C are heated to 45° C. and mixed until homogeneous. The mixed Phases B and C are mixed with Phase A. The pH is adjusted to 7.0. The resulting composition is de-aerated and poured into appropriate containers. The sunscreen shows good skin adhesion and waterproofness.

### Example 13

### Sunscreen/Colorant Composition (Polyderm/Zinc Oxide)

The following ingredients were used:

| | |
|---|---|
| Ultrafine zinc oxide | 47.5 g |
| Deionized water | 1 g |
| Polyderm (BIS-PEG-1 Dimethicone/IPDI Copolymer) (available | 2.5 g |
| from Alzo Int'I, Sayreville, NJ) | |

The ultrafine zinc oxide and water were mixed for 30 seconds in a kitchen-type blender and powder brushed down from the sides of the container and blades before manual mixing. The Polyderm (BIS-PEG-1 Dimethicone/IPDI Copolymer) was added in half portions, with a 30 second mix and brush down after each addition. An additional 30 second mixing was performed after the last addition of Polyderm (BIS-PEG-1 Dimethicone/IPDI Copolymer), with an additional final brush down. The batch was heated overnight in an 80° C. oven. Bulk density of the batch was 10.4 g/cu. in, and moisture content was 0.55%. The batch exhibited hydrophobicity for more than one hour on still water. Spreadability of the product was very high and adhesiveness of the product to skin under running water was moderately high. The skin feel was moderately soft.

### Example 14

### Sunscreen/Colorant Composition (Polyderm/Zinc Oxide)

The following ingredients were used:

| | |
|---|---|
| Ultrafine zinc oxide | 45 g |
| Deionized water | 2 g |
| Polyderm (BIS-PEG-1 Dimethicone/IPDI Copolymer) (available | 5 g |
| from Alzo Int'I, Sayreville, NJ) | |

The ultrafine zinc oxide and water were mixed for 30 seconds in a kitchen-type blender and powder brushed down from the sides of the container and blades before manual mixing. The Polyderm (BIS-PEG-1 Dimethicone/IPDI Copolymer) was added in half portions, with a 30 second mix and brushdown after each addition. An additional 30 second mixing was performed after the last addition of Polyderm (BIS-PEG-1 Dimethicone/IPDI Copolymer), with an additional final brushdown. The batch was heated overnight in an 80° C. oven. Bulk density of the batch was 15.3 g/cu. in, and moisture content was 0.38%. The batch exhibited hydrophobicity for more than one hour on still and shaken water.

### Example 15

The material made according to Example 14 is made into a sunscreen according to the following formula:

| | % w/w |
|---|---|
| Phase A | |
| Pure Water | 68.00 |
| Glycerin (Glycon G100 (99.5%), available from Lonzo Inc.) | 10.00 |
| Material made in Example 14 | 8.00 |
| Preservative (Propylparaben, available from Protameen | q.s. |
| Chemical Inc.) | |
| Phase B | |
| BASE O/W 097 (emulsifying base; available from Sensient | 8.00 |
| Cosmetic Technologies, (LCW), South Plainfield, NJ) | |
| Phase C | |
| COVASTEROL (a plant-based lanolin substitute; available from | 2.00 |
| Sensient Cosmetic Technologies, (LCW), South Plainfield, NJ) | |
| SQUATOL S (a hydrogenated polyisobutene; available from | 4.00 |
| Sensient Cosmetic Technologies, (LCW), South Plainfield, NJ) | |
| | 100.00 |

Phase A components are heated to 40° C. and mixed using a propeller blade mixer until homogenous. Phase B and C are heated to 45° C. and mixed until homogeneous. The mixed Phases B and C are mixed with Phase A. The pH is adjusted to 7.0. The resulting composition is de-aerated and poured into appropriate containers. The sunscreen shows good skin adhesion and waterproofness.

### Example 16

### Colorant Composition (Polyderm/Black Iron Oxide)

The following ingredients were used:

| | |
|---|---|
| Unipure Black LC 989 Iron Oxide (available from Sensient | 47.5 g |
| Cosmetic Technologies, (LCW), South Plainfield, NJ) | |
| Deionized water | 1 g |
| Polyderm (BIS-PEG-1 Dimethicone/PEG-2 Soyamine/IPDI | 2.5 g |
| Copolymer) (available from Alzo Int'I, Sayreville, NJ) | |

The black iron oxide and water were mixed for 30 seconds in a kitchen-type blender and powder was brushed down from the sides of the container and blades before manual mixing. The Polyderm (BIS-PEG-1 Dimethicone/PEG-2 Soyamine/IPDI Copolymer) was added in thirds, with a 30 second mix and brushdown after each addition. The batch was heated overnight in an 80° C. oven. The composition showed good hydrophobicity on still water for one hour.

### Example 17

### Performance of Treated Black Iron Oxide in Mascara

| | % w/w |
|---|---|
| Phase A | |
| Pure Water | 46.70 |
| COVACRYL RH (acrylic film-forming texturing agent; available from | 0.10 |
| Sensient Cosmetic Technologies, (LCW), South Plainfield, NJ) | |
| FUCOMER (seaweed extract; available from Sensient Cosmetic | 1.00 |
| Technologies, (LCW), South Plainfield, NJ) | |
| Methyl Paraben | 0.30 |
| Phase B | |
| Composition made in Example 16 | 12.00 |
| COVABEAD PMMA (spheres of poly(methyl methacrylate); available | 2.00 |
| from Sensient Cosmetic Technologies (LCW), South Plainfield, NJ) | |
| Phase C | |
| BASE O/W 097 (emulsifying base; available from Sensient Cosmetic | 4.50 |
| Technologies, (LCW), South Plainfield, NJ) | |
| Candelilla wax | 2.50 |
| COVALIP 99 (colorless base of waxes, esters and polymers; available | 1.50 |
| from Sensient Cosmetic Technologies, (LCW), South Plainfield, NJ) | |
| SILAMER (Phenyl trimethicone & cetyl PEG/PPG-10/1 dimethicone & | 1.30 |
| polyglyceryl-2-isostearate & hexyl laurate, available from Sensient | |
| Cosmetic Technologies (LCW), South Plainfield, NJ) | |
| Propyl Paraben | 0.30 |
| Phase D | |
| FIBERLON Y10 (nylon fibers; available from Sensient Cosmetic | 0.30 |
| Technologies, (LCW), South Plainfield, NJ) | |
| Phase E | |
| Pure Water | 10.00 |
| PPG-17/IPDI/DMPA | 3.00 |
| COVACRYL E14 (acrylate copolymer; available from Sensient | 11.00 |
| Cosmetic Technologies, (LCW), South Plainfield, NJ) | |
| COVACRYL A15 (acrylate copolymer; available from Sensient | 3.50 |
| Cosmetic Technologies, (LCW), South Plainfield, NJ) | |
| | 100.00% |

Phase A: Water is heated in main vessel to 70° C., the components of Phase A are added one by one using a propeller mixer until dissolved. Phase B is slowly added to Phase A, homogenizing under a high speed mixer until a good dispersion of the colors is achieved. The temperature is maintained at 70° C. The components of Phase C are dissolved together at 80° C. until homogeneous and maintained at that temperature. Phase C is added to the main vessel, slowly blending with a propeller blade mixer for 15 minutes at 80° C. Phase D components are mixed until homogenous. The main vessel is cooled to 40° C. Phase D is slowly added to main vessel with continued stirring, maintaining the temperature at 40° C. The formulation is cooled to an appropriate pouring temperature. The mascara formulation shows good long-wearing properties.

### Example 18

### Colorant Composition (Polyderm/Red Iron Oxide)

The following ingredients were used:

| | |
|---|---|
| Unipure Red LC 383 Iron Oxide | 47.5 g |
| Deionized water | 1 g |
| Polyderm (BIS-PEG-1 Dimethicone/PEG-2 Soyamine/IPDI | 2.5 g |
| Copolymer) (available from Alzo Int'I, Sayreville, NJ) | |

The red iron oxide and water were mixed for 30 seconds in a kitchen-type blender and powder was brushed down from the sides of the container and blades before manual mixing. The Polyderm (BIS-PEG-1 Dimethicone/PEG-2 Soyamine/IPDI Copolymer) was added in thirds, with a 30 second mix and brushdown after each addition. The batch was heated overnight in an 80° C. oven. The pigment showed good hydrophobicity on still water for one hour.

### Example 19

### Hydrophobicity and Lipophobicity Tests of Treated Colorants

The compositions made in Examples 3 and 18, and untreated Unipure Red LC 383 Iron Oxide (used as an ingredient in each of Examples 3 and 18) were made into pressed powders at a pressure of 400 psi, using 4.00 grams of material in a 2.00 cm diameter metal pan, and a 10 seconds dwell time.

The following fluids were each applied as a drop to the surface of each of the pressed powders: water, Dow Corning™ 245 Fluid, caprylic/capric triglyceride and castor oil. The time taken for the drop of fluid to be absorbed into the pressed powder was monitored. The results are shown below:

| | Untreated | | |
|---|---|---|---|
| | Unipure Red | Composition Made | Composition Made |
| | LC 383 | in Example 3 | in Example 18 |
| Water | Absorbed fast | Absorbed slowly - | Absorbed slowly - |
| | in 6 seconds | more than 10 min | more than 10 min |
| | (Hydrophilic) | (Hydrophobic) | (Hydrophobic) |
| Dow | Absorbed fast | Absorbed fast in | Absorbed fast in |
| Corning ™ | in 7 seconds | 12 seconds | 10 seconds |
| 245 Fluid | (Lipophilic) | (Lipophilic) | (Lipophilic) |
| Caprylic/ | Absorbed | Absorbed slowly in | Absorbed slowly in |
| Capric | slowly in | 1 min 47 sec | 1 min 32 sec |
| Triglyceride | 32 seconds | (Lipophobic) | (Lipophobic) |
| | (Lipophobic) | | |
| Castor Oil | Absorbed | Absorbed slowly - | Absorbed slowly - |
| | slowly in | more than 10 min | more than 10 min |
| | 8 min 31 sec | (Lipophobic) | (Lipophobic) |
| | (Lipophobic) | | |

The compositions made in examples 3 and 18 were much more hydrophobic than the untreated Unipure Red LC 383 colorant. Water was absorbed, if at all, over a much longer period for the treated colorants. The compositions made in examples 3 and 18 also resisted wetting by Dow Corning™ 245 Fluid, caprylic/capric triglyceride and castor oil to a greater extent than untreated Unipure Red LC 383 colorant.

### Example 20

### Transfer Resistance of Treated Colorants Adhered to the Skin

The compositions made in Examples 3 and 18, and untreated Unipure Red LC 383 Iron Oxide (used as an ingredient in each of Examples 3 and 18) were made into pressed powders at a pressure of 400 psi, using 2.00 grams of material each in a 1.25 inch diameter metal pan. The initial weight of paper, brush and each of the pressed powders were recorded. The paper was placed underneath the pressed powders to catch powder residue, and the pressed powders were each brushed with 10 strokes of brush. The brush and paper were weighed to determine how much powder had adhered to the brush, and how much residue had dusted onto the paper. The results are shown below:

| | Powder | Powder | |
|---|---|---|---|
| | Adhesion | Residue | |
| | on | on | |
| | Brush (g) | Paper (g) | OBSERVATION |
| Untreated | 0.02 | 0.03 | Most powder residue on paper and |
| Unipure Red | | | less transferred to the brush. |
| LC 383 | | | |
| Colorant | | | |
| Composition | 0.02 | <0.01 | More powder adheres on the brush |
| of Example | | | and less powder residue on the |
| 3 | | | paper (high adhesion to the brush |
| | | | is suggestive of a high adhesion to |
| | | | the skin) |
| Composition | <0.01 | 0.01 | Less powder was transferred to the |
| of Example | | | brush, minimal powder residue on |
| 18 | | | paper. |

The powder that adhered to the brush for each sample was then brushed on the skin using 10 strokes. A clean sheet of tissue paper was pressed on the skin, and assessed visually to determine the transfer resistance of the colorants from the skin.

For the untreated Unipure Red LC 383 Iron Oxide colorant, a lot of colorant transferred from the skin to the tissue paper. The tissue paper was marked with more color than that used in the tests of either of the treated colorants, indicating that the untreated colorant was not resistant to transfer.

For the composition of Example 3, very little of the treated colorant was transferred from the skin to the tissue paper. Only very faint color was noted on the tissue paper, indicating that the treated colorant was much more resistant to transfer than the untreated colorant.

For the composition of Example 18, while little colorant was transferred to the skin from the brush (since little had been transferred to the brush), very little colorant transferred from the skin to the tissue paper.

### Example 21

### Colorant Composition For Application to Hair

The following formulation is used to apply pigments to the hair:

| | % w/w |
|---|---|
| Phase A | |
| Deionized Water | 29.0 |
| BASE O/W 097 (emulsifying base, available from Sensient | 3.00 |
| Cosmetic Technologies (LCW), South Plainfield, NJ) | |
| Phase B | |
| Propylene Glycol (Propylene Glycol USP/EP, available from | 9.00 |
| Dow Chemical) | |
| UNIPURE Black LC 989 Iron Oxide treated with Polyderm LSA | 15.00 |
| (BIS-PEG-1 Dimethicone/PEG-2 Soyamine/IPDI CoPolymer) | |
| TALC OOC treated with Polyderm LSA | 3.00 |
| Phase C | |
| Deionized Water | 15.00 |
| Avalure UR-450 (urethane polymer dispersion, available from | 2.00 |
| Noveon) | |
| COVACRYL E14 (available from Sensient Cosmetic | 5.00 |
| Technologies (LCW), South Plainfield, NJ) | |
| Polyglycerol-3 Laurate (Hydramol TGL, available from Noveon, | 1.00 |
| Inc.) | |
| Propylene Glycol (Propylene Glycol USP/EP, available from | 2.00 |
| Dow Chemical) | |
| Phase D | |
| COVACRYL MS11 (available from Sensient Cosmetic | 14.00 |
| Technologies (LCW), South Plainfield, NJ) | |
| COVAPLAST (available from Sensient Cosmetic Technologies | 1.00 |
| (LCW), South Plainfield, NJ) | |
| Phase E | |
| Preservative Germaben II (propylene glycol, diazolidinyl urea, | 1.00 |
| methylparaben and propylparabens, available from Sutton | |
| Laboratories) | |
| | 100.00 |

Phase C components are mixed using a propeller blade until homogeneous. For Phase B, the Unipure Black LC color and talc are mixed and dispersed in propylene glycol using a three roller mill. Base O/W 097 of phase A is heated to 45° C. until liquefied; the water of phase A is added and mixed in. Phase D components are mixed together. Phases A, B, C and D are combined, one at a time, in the order recited and mixed thoroughly. The preservative of phase E is added and mixed. The composition is placed in appropriate containers.

The composition is applied to hair as a hair colorant.

### Example 22

### Performance of Treated Materials in a Cosmetic Pressed Powder

The following colorants were surface treated as described below, all available from Sensient Cosmetic Technologies/LCW, South Plainfield, N.J.: titanium Dioxide (Atlas White) and iron oxides (Unipure Red LC 383, Unipure Yellow LC 182, Unipure Black LC 989).

Colorant Compositions (Polyderm/cosmetic colorant)

The following ingredients were used:

| | |
|---|---|
| Cosmetic Colorant | 48.5 g |
| Deionized water | 1 g |
| Polyderm LSA (BIS-PEG-1 Dimethicone/PEG-2 Soyamine/IPDI | 1.5 g |
| Copolymer) (available from Alzo Int'l, Sayreville, NJ) | |

The cosmetic colorant and water were mixed for 30 seconds in a kitchen-type blender and powder was brushed down from the sides of the container and blades before manual mixing. The Polyderm LSA (BIS-PEG-1 Dimethicone/PEG-2 Soyamine/IPDI Copolymer) was added in thirds, with a 30 second mix and brushdown after each addition. The batch was heated overnight in an 80° C. oven. This procedure was repeated individually for the titanium dioxide and the red, yellow and black iron oxides. The pigments all showed good hydrophobicity on still water for one hour. The pigments were then formulated into a cosmetic pressed powder as shown below:

| | % w/w |
|---|---|
| Phase A | |
| TALC 141 | 79.8 |
| Magnesium stearate (available from Spectrum Chemical) | 2.00 |
| Preservative: Propylparaben (available from Protameen | 0.20 |
| Chemical Inc.) | |
| Phase B | |
| ATLAS WHITE TITANIUM DIOXIDE 3% Polyderm LSA | 6.00 |
| (BIS-PEG-1 Dimethicone/PEG-2 Soyamine/IPDI Copolymer) | |
| RED IRON OXIDE UNIPURE LC 383 3% Polyderm LSA | 2.00 |
| (BIS-PEG-1 Dimethicone/PEG-2 Soyamine/IPDI Copolymer) | |
| YELLOW IRON OXIDE LC 182 3% Polyderm LSA | 1.70 |
| (BIS-PEG-1 Dimethicone/PEG-2 Soyamine/IPDI Copolymer) | |
| BLACK IRON OXIDE LC 989 3% Polyderm LSA | 0.30 |
| (BIS-PEG-1 Dimethicone/PEG-2 Soyamine/IPDI Copolymer) | |
| Phase C | |
| COVAPRESS TW 966 (binding agent; available from | 8.00 |
| Sensient Cosmetic Technologies (LCW), South Plainfield, NJ) | |
| | 100.00% |

Weigh Phase A and B and place the powders in a blender. Process until color develops. Add Phase C and process again for several minutes. Press at 380 psi with a 1 minute dwell time. The formulation was compared against a formulation made with untreated pigments. The formulation containing the treated pigments did not crack after three drops of the pressed powder from a height 12″ while that containing untreated pigments did crack at the third drop. This test demonstrated that the treatment contributed better cohesiveness to the compacted powder.

### Example 23

### Liquid Foundation

The red iron oxide and titanium dioxide surface treated as described in example 21 was formulated into a liquid foundation formula as shown below.

| | W/W % |
|---|---|
| Phase A | |
| SILAMER (Phenyl trimethicone & cetyl PEG/PPG-10/1 | 15.00 |
| dimethicone & polyglyceryl-2-isostearate & hexyl laurate | |
| (available from Sensient Cosmetic Technologies (LCW), | |
| South Plainfield, NJ) | |
| Cetyl Dimethicone (Abil Wax 9801; available from Degussa | 1.90 |
| Corp., Parsippany, NJ) | |
| Synthetic Beeswax | 0.65 |
| Hydrogenated Castor oil (castor wax; available from | 0.50 |
| CasChem) | |
| Hydrogenated Castor oil (castor wax; available from | 2.70 |
| CasChem) | |
| Phase B | |
| Titanium dioxide (ATLAS WHITE with 3% Polyderm LSA) | 7.50 |
| RED IRON OXIDE (Unipure Red LC 383 with 3% Polyderm | 1.55 |
| LSA) | |
| LUMIRAL (light diffusing pigment; available from Sensient | 1.20 |
| Cosmetic Technologies (LCW), South Plainfield, NJ) | |
| COVABEAD GLASS (spherical powder; available from | 4.60 |
| Sensient Cosmetic Technologies (LCW), South Plainfield, NJ) | |
| Phase C | |
| Dow Corning 245 (cyclomethicone, available from Dow | 10.00 |
| Corning) | |
| Phase D | |
| D.I. Water | 47.60 |
| Polyglycerol-3 Laurate (Hydramol TGL, available from | 0.40 |
| Noveon Inc.) | |
| Propylene Glycol (Propylene Glycol USP/EP, available from | 6.00 |
| Dow Chemical) | |
| Preservative: Germaben II (propylene glycol, diazolidinyl | 0.40 |
| urea, methylparaben and propylparabens, available from | |
| Sutton Laboratories) | |
| | 100.00% |

Phase A was blended and heated with mixing to 80° C. Phase B was then blended in an Osterizer until homogenous. Phase B was added to A with mixing, then Phase C was added to the bulk with mixing. The entire mixture was blended for 30 minutes using a Silverson mixer at 800 rpm until homogenous. Phase D was then added to the bulk at a temperature of 75° C. The formulation was then homogenized until it cooled to 50° C. The formulation was then cooled down and deaerated before pouring into a container. This formulation was compared to a similar formulation made with untreated titanium dioxide and red iron oxide. After storage in an oven for four weeks, the viscosity of the formulation made with treated pigments was 16,200 cps, as compared to 17,300 cps for the formulation made with the untreated pigments. The formulation made with treated pigments was also less thixotropic than that made with untreated pigments.

### Example 24

### Performance of Treated Black Iron Oxide in Mascara

| | % w/w |
|---|---|
| Phase A | |
| Synthetic Beeswax (Koster Keunen, LLC) | 6.50 |
| Siliconyl Candellila Wax # 223 (Koster Keunen, LLC) | 1.50 |
| Glyceryl stearate (GMS 450) (Lipo Chemicals) | 2.75 |
| Mica (Submica E, available from Sensient Cosmetic | 0.50 |
| Technologies (LCW), South Plainfield, NJ) | |
| Sorbitan Sesquioleate (Jeen Chemicals) | 1.70 |
| Stearic Acid (Emersol 132 NF) (Cognis Corporation) | 0.50 |
| Paraffin-wax # 150/155 (Koster-Keunan, LLC) | 0.50 |
| Preservative: Propylparaben (available from Protameen | 0.30 |
| Chemical Inc.) | |
| Phase B | |
| Black Iron oxide as described in example 22 | 5.00 |
| Phase C | |
| Deionized water | 69.75 |
| Polyglycerol-3 Laurate (Hydramol TGL, available from | 0.75 |
| Noveon Inc.) | |
| PEG 8 (Dow Chemical Company) | 1.00 |
| Propylene Glycol (Propylene Glycol USP/EP, available | 1.00 |
| from Dow Chemical) | |
| COVACRYL P12 (acrylate copolymer; available from | 0.50 |
| Sensient Cosmetic Technologies, (LCW), South | |
| Plainfield, NJ) | |
| Phase D | |
| Isododecane (Permethyl 99A) (Presperse) | 7.75 |
| | 100.00% |

Phase A: Combine and heat in the main vessel to 80° C. and mix until homogeneous. Prepare Phase C, heat to 30 C and mix until homogeneous. Add Phase B to Phase A and continue mixing until the pigment is dispersed. Slowly add Phase C to the bulk until emulsified. Mix for 5 minutes. Cool to 50 C, and add Phase D to the bulk. Mix for another five minutes, then put into an appropriate container. The formulation has a 2 minute dry time after applied to the eyelashes.

### Example 25

Collagen sheets (SpaElegance, Bridgeville, Pa.) were used as a substitute for human skin to compare adhesion of black iron oxide treated as described in Example 21 and with 3% Polyderm PPI-SI-L2 to untreated black iron oxide. A 1.25″×1.25″ piece of collagen was placed together with 1.00 gram of test powder into a 60 mL bottle and capped. The bottle was then manually shaken for 15 seconds and the piece of collagen recovered and weighed. A quantity of 0.9% of the untreated powder adhered to the collagen, while a quantity of 2.4% of the treated powder adhered to the collagen. A powder treated with 3% Polyderm L2 had an adhesion of 2.9%

While the present invention has now been described and exemplified with some specificity, those skilled in the art will appreciate the various modifications, including variations, additions and omissions, that may be made in what has been described. All patents, publications and references cited herein are hereby fully incorporated by reference. In case of conflict between the present disclosure and incorporated patents, publications and references, the present disclosure should control.

1. A composition comprising a colorant surface-treated with an effective amount of a silicone polyurethane.

2. The composition of claim 1, wherein the colorant comprises between about 3% to about 5% of a silicone polyurethane.

3. The composition of claim 1, wherein the silicone polyurethane comprises at least one of: a dimethicone/isophorone diisocyanate copolymer, a dimethiconol/isophorone diisocyanate copolymer, polydimethyl siloxane-PEG ether/isophorone diisocyanate copolymer, polydimethyl siloxane-PPG ether/isophorone diisocyanate copolymer, and a combination thereof.

4. The composition of claim 3, wherein the ratio of silicone to diisocyanate is from about 1:20 to about 20:1.

5. The composition of claim 3, wherein the ratio of dimethicone to isophorone diisocyanate is from about 2:1 to about 16:1.

6. The composition of claim 3, wherein the composition comprises a dimethiconol/isophorone diisocyanate copolymer and the dimethiconol/isophorone diisocyanate copolymer comprises at least one of: dimethiconol/isophorone diisocyanate copolymer 50%, dimethiconol-PEG-2 soyamine/isophorone diisocyanate copolymer, dimethicone copolyol/isophorone diisocyanate copolymer, dimethicone/isophorone diisocyanate copolymer, dimethicone/PEG-2 soyamine/isophorone diisocyanate copolymer, and a combination thereof.

7. The composition of claim 1, wherein the silicone polyurethane comprises a siloxane containing the following repeating structure and also containing a reactive hydroxyl group: 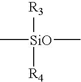
wherein R₃ and R₄ are each, independently, alkyl or aryl.

8. The composition of claim 7, wherein R₃ and R₄ may represent the same group or different groups.

9. The composition of claim 7, wherein R₃ and R₄ may independently be a group selected from: methyl, ethyl, propyl, phenyl, methylphenyl and phenylmethyl, alpha olefin, allyl alcohol alkoxylated, fluoro compounds and allyl alcohols.

10. The composition of claim 7, wherein the siloxane comprises at least one of: polydimethyl siloxane, polydiethylsiloxane, polymethylphenylsiloxane and a combination thereof.

11. The composition of claim 1, wherein the silicone polyurethane has the structure: 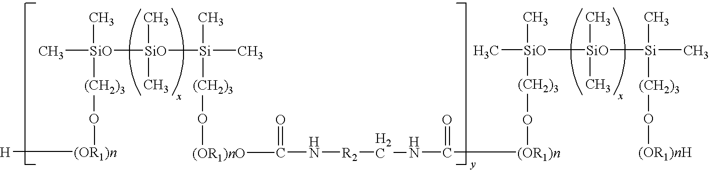
wherein:
R₁ is a C2 to C8 alkylene group and n is about 2-200;
R₂ is a C2 through C36 linear, cyclic or branch-chained saturated or unsaturated hydrocarbon group which is substituted or unsubstituted, monomeric or dimeric, an aromatic group, saturated, unsaturated, aromatic or halogen substituted, linear, cyclic, aromatic or branch chained hydrocarbons;
y is an integer of about 1-1000; and
x is an integer of about 1-9000.

12. The composition of claim 1, wherein the silicone polyurethane has the structure: 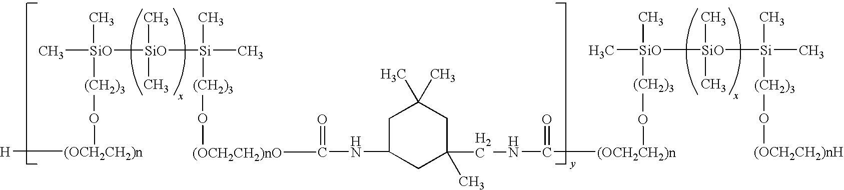
wherein:
y is an integer of about 1-1000;
x is an integer of about 1-9000; and
n is about 2-200.

13. The composition of claim 1, wherein the silicone polyurethane has a molecular weight of from about 2,000 to about 10,000,000.

14. The composition of claim 1, wherein the silicone polyurethane has a molecular weight of from about 5,000 to about 1,000,000.

15. The composition of claim 1, further comprising a tertiary amine, a monohydric alcohol, an alkanolamine fatty acid ester, castor oil, or combinations thereof.

16. The composition of claim 1, wherein the colorant comprises at least one of a color extender, a dye, an inorganic colorant, a natural inorganic pigment, a synthetic inorganic pigment, an organic colorant, a synthetic organic lake, a toner, and a combination thereof.

17. The composition of claim 16, wherein the colorant comprises an inorganic colorant, and the inorganic colorant comprises at least one of: titanium dioxide, manganese violet (CI 77742), ultramarine blue, pink or violet (CI 77007), chromium oxide (CI 77288), chromium hydroxide, hydrated chromium oxide (CI 77289), ferric ferrocyanide, ferric blue (CI-77510), zinc oxide, zirconium dioxide, natural or synthetic iron oxides, talc, zeolite, kaolin and combinations thereof.

18. The composition of claim 16, wherein the colorant comprises an organic colorant, and the organic colorant comprises at least one of: carbon black, carmine, phthalocyanine blue and green pigment, diarylide yellow and orange pigments, azo-type red and yellow pigments, toluidine red, litho red, naphthol red and brown pigments, lakes of chlorophyllin copper complex, black carrot extract (anthrocyanin), beet root extracts, riboflavin, curcumin, azo, indigoid, triphenylmethane, anthraquinone, bromo, fluorescein and xantheine dyes, dyes which are designated as D&C and FD&C blues, browns, greens, oranges, reds, yellows, natural or synthetic melanin, insoluble metallic salts of certified color additives and combinations thereof.

19. The composition of claim 16, wherein the colorant comprises a color extender, and the color extender comprises at least one of: a white or non-pigmentitious powder, selected from: bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, and mixtures thereof.

20. The composition of claim 1, further comprising a moistening agent, a dissolving agent, an emulsifiying agent, a lanolin substitute, a lubricant, a solvent, a moisturizing agent, a humectant, a fragrance carrier or a combination thereof.

21. A cosmetic product comprising the composition of claim 1.

22. The cosmetic product of claim 21, wherein the cosmetic product is a mascara, a pressed powder make-up, a liquid make-up, a lipstick, a skin care, or a sunscreen.

23. The cosmetic product of claim 21, further comprising a moistening agent, a dissolving agent, an emulsifiying agent, a lanolin substitute, a lubricant, a solvent, a moisturizing agent, a humectant, a fragrance carrier or a combination thereof.

24. A personal care product comprising the composition of claim 1.

25. The personal care product of claim 24, wherein the personal care product is a lotion, a cream, a gel, a toothpaste, a sunscreen, or combinations thereof.

26. The personal care product of claim 24, further comprising a moistening agent, a dissolving agent, an emulsifiying agent, a lanolin substitute, a lubricant, a solvent, a moisturizing agent, a humectant, a fragrance carrier or a combination thereof.

27. A hair colorant comprising the composition of claim 1.

28. The hair colorant of claim 27, further comprising a moistening agent, a dissolving agent, an emulsifiying agent, a lanolin substitute, a lubricant, a solvent, a moisturizing agent, a humectant, a fragrance carrier or a combination thereof.

29. A method for making a surface-treated colorant, the method comprising: surface-treating a colorant with an effective amount of a silicone polyurethane to form a surface-treated colorant.

30. The method of claim 29, further comprising mixing the surface-treated colorant with a moistening agent, a dissolving agent, an emulsifiying agent, a lanolin substitute, a lubricant, a solvent, a moisturizing agent, a humectant, a fragrance carrier or a combination thereof.

31. The method of claim 29, wherein the surface-treating comprises spraying or atomizing the silicone polyurethane.

32. The method of claim 29, wherein surface-treating comprises agitating the colorant by air or by mechanical means.

33. The method of claim 29, wherein the silicone polyurethane to colorant ratio is about 15 parts silicone polyurethane to about 85 parts colorant to about 1 part surface treatment to about 99 parts colorant.

34. The method of claim 29, wherein the silicone polyurethane to colorant ratio is about 10 parts silicone polyurethane to about 90 parts colorant to about 1 part surface treatment to about 99 parts colorant.

35. The method of claim 29, wherein the surface treatment comprises water in the ratio of about 1 part water to about 1 part surface treatment by weight up to about 1 part water to about 10 parts surface treatment by weight.

36. The method of claim 29, further comprising drying the surface-treated colorant.

37. A method of making a surface-treated colorant product, comprising incorporating the surface treatment colorant formed according to claim 29 into a cosmetic, skin care product, a personal care product, a sunscreen, a nail lacquer or a hair colorant.

38. A method of improving the hydrophobicity or the adherence to the surface of skin of a colorant, the method comprising: surface-treating a colorant with an effective amount of a silicone polyurethane to form a surface-treated colorant having an improved hydrophobilicy or an improved adherence to a skin surface compared to the colorant before surface-treatment.

39. The method of claim 38, wherein the surface-treated colorant having improved hydrophobicity repels water for more than about 30 seconds.

40. The method of claim 38, wherein the surface-treated colorant having improved hydrophobicity repels water for more than about 10 minutes.
